(19) United States
(12) Patent Application Publication
(10) Pub. No.: US 2004/0204619 A1
(43) Pub. Date: Oct. 14, 2004

(54) Method for producing isopropyl chloride for use as a foam-blowing agent

(75) Inventors: Eric L. Mainz, Goddard, KS (US); Richard L. Wilson, Mulvane, KS (US); John Lee Dawkins, Derby, KS (US); Janice M. Nyberg, Wichita, KS (US)

Correspondence Address: ARMSTRONG, KRATZ, QUINTOS, HANSON & BROOKS, LLP, Intellectual Property Law Offices, Suite 220, 502 Washington Avenue, Towson, MD, 21204, US

(73) Assignee: VULCAN CHEMICALS a Business Group of Vulcan Materials Company

(21) Appl. No.: 10/809,633

(22) Filed: Mar. 25, 2004

Related U.S. Application Data
(60) Provisional application No. 60/462,599, filed on Apr. 11, 2003.

(51) Int. Cl. C07C 17/093

(52) U.S. Cl. 570/234

(57) ABSTRACT

Isopropyl chloride (IPC) is prepared by the chlorination of propane or propene or propyne, or by any other means resulting in an IPC product containing significant amounts of undesired olefins. Such impure product may be purified by chlorination followed by distillation, by distillation followed by chlorination followed by distillation, by chlorination followed by treatment to reduce chlorine concentration followed by distillation, or by distillation followed by chlorination followed by treatment followed by distillation.

### CROSS REFERENCE TO RELATED APPLICATIONS

This application is related to provisional patent application Serial No. 60/462,599, filed Apr. 11, 2003 titled “Method for Producing Isopropyl Chloride for Use as a Foam-Blowing Agent”.

### BACKGROUND OF THE INVENTION

1. Field of the Invention

The present invention relates to the preparation of foam-blowing agents and particularly, the preparation of purified isopropyl chloride, a foam-blowing agent for the preparation of polyurethane foam.

2. Description of Related Art

Isopropyl chloride is useful as a blowing agent to prepare polyurethane foam. Processes are available that are capable of producing an isopropyl chloride product with excellent purity. For example, isopropyl alcohol may be reacted with hydrogen chloride. Alternately, propene may be hydrochlorinated in the presence of ferric chloride catalyst. Such processes, while efficient in absolute terms, may be less economical compared to recovering purified isopropyl chloride from waste streams or byproduct streams originating in processes primarily intended to produce other materials.

For example, the chlorination of propane ordinarily produces a mixture of products, including isopropyl chloride. Propene is reacted with chlorine at high temperature to produce allyl chloride and a variety of other chlorinated propenes and propanes, including isopropyl chloride. The commercial manufacture of allyl chloride results in the formation of waste or byproduct streams that contain significant amounts of isopropyl chloride together with such chlorinated propenes and propanes as cis-1-chloropropene, trans-1-chloropropene, 2-chloropropene, allyl chloride, 1,2-dichloropropane, and 1-chloropropane. The monochloropropenes, in particular, may be difficult to separate from isopropyl chloride by simple distillation. The commercial manufacture of 1,3-dichloropropene, a soil fumigant, can also result in the formation of waste or byproduct streams that contain significant amounts of isopropyl chloride and other chlorinated propanes and chlorinated propenes which may be difficult to separate from isopropyl chloride by simple distillation.

Hence, a method is desired to remove chlorinated olefins from a starting material containing chlorinated olefins in combination with isopropyl chloride and other impurities.

### SUMMARY OF THE INVENTION

It is an object of the present invention to prepare purified isopropyl chloride from a starting material containing impure isopropyl chloride and olefinic impurities.

It is a further object of the invention to prepare the purified isopropyl chloride by reacting the impure mixture with a chlorinating agent followed by distillation.

In accordance with the teachings of the present invention, there is disclosed a method to prepare purified isopropyl chloride from a starting material containing isopropyl chloride and impurities that are relatively easy to oxidize compared to isopropyl chloride. A chlorinating agent is reacted with the starting material to produce a mixture containing isopropyl chloride and chlorinated impurities. The mixture is distilled to separate isopropyl chloride from the chlorinated impurities.

In further accordance with the teachings of the present invention, there is disclosed a method to prepare purified isopropyl chloride from a starting material containing isopropyl chloride and impurities that are relatively easy to oxidize compared to isopropyl chloride. The starting material is distilled to produce a fraction enriched in isopropyl chloride. The enriched fraction is reacted with a chlorinating agent to produce a mixture containing isopropyl chloride and chlorinated impurities. The mixture is distilled to separate purified isopropyl chloride from the chlorinated impurities.

In still further accordance with the teachings of the present invention, there is disclosed a method to prepare purified isopropyl chloride from a starting material containing isopropyl chloride and impurities that are relatively easy to oxidize compared to isopropyl chloride. The starting material is reacted with a chlorinating agent to produce a mixture containing isopropyl chloride and chlorinated impurities. The product mixture is treated to produce a mixture containing reduced chlorine concentration. The treated product mixture is distilled to separate purified isopropyl chloride from the chlorinated impurities.

In yet another aspect, there is disclosed a method to prepare purified isopropyl chloride from a starting material containing isopropyl chloride and impurities that are relatively easy to oxidize compared to isopropyl chloride. The starting material is distilled to produce a fraction enriched in isopropyl chloride. The enriched fraction is reacted with a chlorinating agent to produce a mixture containing isopropyl chloride and chlorinated impurities. The product mixture is treated to produce a treated mixture containing reduced chlorine concentration. The treated mixture is distilled to separate purified isopropyl chloride from the chlorinated impurities.

These and other objects of the present invention will become apparent from a reading of the following specification taken in conjunction with the enclosed drawings.

### BRIEF DESCRIPTION OF THE DRAWINGS

FIG. 1 is a diagram showing reaction followed by distillation.

FIG. 2 is a diagram showing distillation followed by reaction.

FIG. 3 is a diagram showing reaction, treatment and distillation.

FIG. 4 is a diagram showing distillation, reaction, treatment and distillation.

### DESCRIPTION

It has been found that starting materials such as chlorinated olefins, isopropyl chloride and other impurities may be treated with a chlorinating agent such as chlorine, to convert olefinic impurities to chlorinated alkanes that may be more easily separated from isopropyl chloride. In one method, the starting material is directly chlorinated, with or without a catalyst, in liquid or vapor phase, under conditions effective to accomplish the desired reactions and then the chlorinated product is distilled to recover purified isopropyl chloride. Under an alternative scenario, first distill the starting material to recover a mixture enriched in isopropyl chloride, then chlorinate the enriched mixture to convert undesirable olefins to chlorinated alkanes, and then distill again to separate the purified isopropyl chloride.

Any effective catalyst may be employed to improve the kinetics or the selectivity of the chlorination step. Effective catalysts include, but are not limited to, the following: light, ferric chloride, other transition metal chlorides, aluminum chloride, antimony chlorides, stannic chloride, zinc chloride, lewis acids, bismuth chloride, gallium chloride, boron chloride, activated carbon, alumina and silica. The chlorination step may be carried out in liquid or vapor phase. Generally, the temperature range of −150° C. and +200° C. will be effective to achieve desirable kinetics with desirable selectivity.

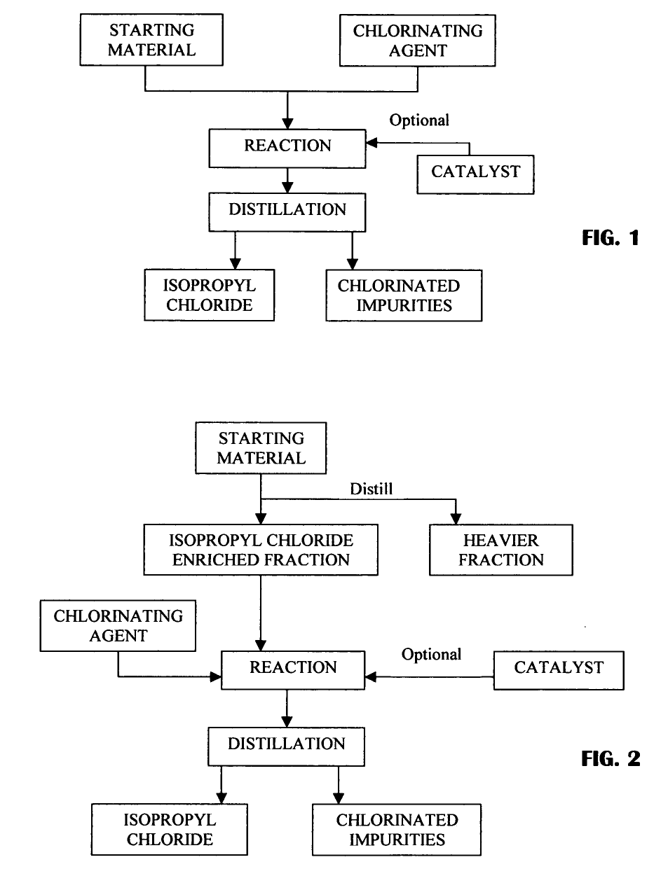

Description of a Process Using Low Grade Feed Stock and No Front-End Distillation (FIG. 1)

A reactor is fed with raw feedstock and chlorine. The reactor is agitated to assure that the reactants are mixed. Reactor construction materials may be Tantalum, Monel™, Inconel™, glass lined steel, or other materials resistant to corrosion by chlorine and hydrogen chloride together. Glass lined steel is preferred for situations where water may be present in the reaction mixture. An option for improving reaction efficiency is to equip the reactor with a system for regulating temperature. An example is a jacketed reactor using a regulated tempered water supply. The reactor may be a continuous reactor or a batch reactor. Either type of reactor has its own advantages and disadvantages.

Chlorination reactions convert chloropropene isomers to trichloropropane according to the reaction: C₃H₅Cl+Cl₂→C₃H₅Cl₃.

To assure substantially complete chloropropene extinction, excess chlorine in the form of a chlorinating agent, is added to the chlorination reactor. The chlorinating agent may be chlorine, antimony pentachloride, phosphorous pentachloride, hypochlorite, hypochlorous acid or other agents known to persons skilled in the art. The excess chlorine is calculated based on the olefin content of the raw feed. The example described later will use 10% excess chlorine feed—which is to say that for every mole equivalent of olefin fed into the reactor, 1.1 moles of chlorine are also introduced. The practical range for excess chlorine feed is 1% to 35% with the most preferred range being 5% to 15%.

Chlorine substitution reactions are also expected. These reactions generate hydrogen chloride and are a cause of inefficiency. One of these reactions is: C₃H₇Cl+Cl₂→C₃H₆Cl₂+HCl.

Chlorine substitution reactions are unavoidable, but can be minimized by using process conditions where the chlorine addition reaction with chloropropene isomers is favored. A reaction temperature between 0° C. and 60° C. is preferred, with the most preferable condition being between 10° C. and 30° C.

Reactor residence time is dependent on reaction temperature and the degree of chloropropene extinction required.

Chlorinated feedstock is drawn from the reactor and fed into a distillation process. Alternatively, the chlorinated feedstock is fed into a second reactor, where the chlorination process is driven further toward completion, then fed into a distillation process. In a process where a secondary reactor is used, the reactor effluent is heated to a temperature between 45° C. and 80° C. and passed through a second reactor with a residence time between 5 minutes and 6 hours. The most preferred range is 50° C. to 60° C. and a residence time between 1 minute and 1 hour. Although an agitated reactor can be used as the secondary reactor, a plug flow reactor or a batch reactor is preferred.

The distillation process will typically use two towers. The first tower will be used to separate hydrogen chloride from isopropyl chloride. Substantially all of the hydrogen chloride from the chlorinated feedstock is recovered in the tower overhead fraction. Most of the isopropyl chloride in the chlorinated feedstock is recovered in the tower bottom fraction.

The second tower will be used to separate isopropyl chloride product from heavier chlorinated hydrocarbons. Most of the isopropyl chloride will be recovered in the overhead fraction. Substantially all of the heavier chlorinated hydrocarbons will be recovered in the bottom fraction.

Description of a Process Whereby Chlorination and Distillation are Completed in a Single Vessel

Raw feedstock having a wide range in isopropyl chloride concentration can be successfully fed to a distillation tower in order to chlorinate the monochloropropenes and effect a separation of isopropyl chloride from the other components. For example, the isopropyl chloride can range in composition from 20% to more than 80%. The other major component, 1,2-dichloropropane can range in concentration from 5% to 80%. A distillation tower is fed with raw feedstock and chlorine. Or the chlorine can be added to the raw feedstock and premixed prior to entering the distillation tower. The mixed feeds can enter the bottom of the tower or at a location near the bottom of the tower. The chlorine reacts with the olefins while in the bottom to intermediate sections of the distillation tower producing a reduced level of monochloropropenes in the mixture. In the lower stages of the tower, the heavier components are separated from the isopropyl chloride and the lower boiling components. If sufficient distillation efficiency is available in the tower, further separation of the components is accomplished in the single distillation step. High purity isopropyl chloride can be drawn off at an upper stage in the tower. Low boiling components including hydrogen chloride, inerts and light hydrocarbons and hydrochlorocarbons having a boiling point less than isopropyl chloride are drawn from the top of the tower. If the isopropyl chloride product drawn from the tower is not of sufficient quality, the material can be fed to a second distillation process for final purification.

The preferred overhead operating pressure of the distillation is 0 to 50 psig with 10-15 psig being the most preferred range. The bottom temperature should be kept below 160° C., preferably below 130° C. to prevent dehydrochlorination of the bottom material. The purity of the isopropyl chloride recovered in this example is about 99.5% dependent on the number of separation stages available in the distillation column.

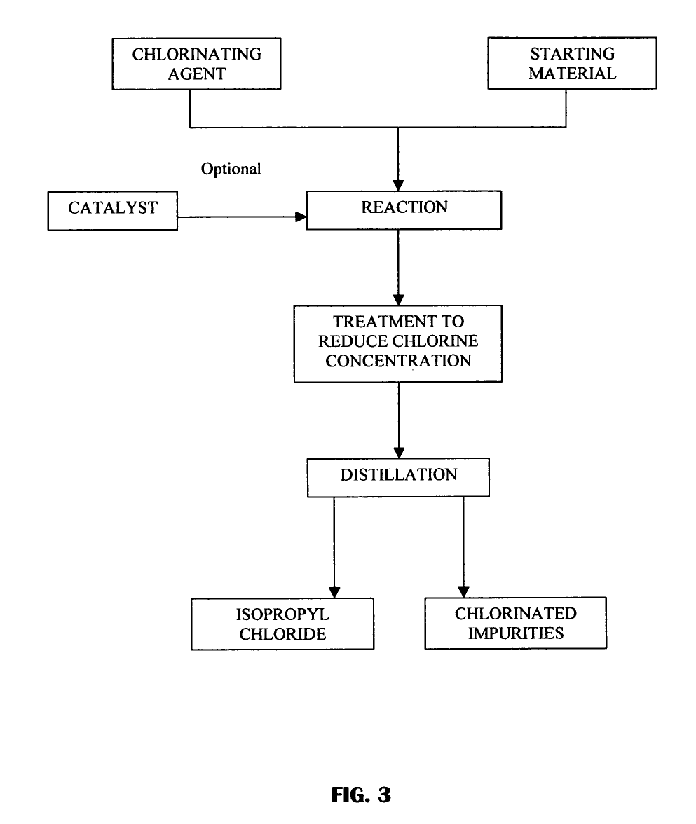

Description of a Process Using a Front-End Distillation to Concentrate Isopropyl Chloride (FIG. 2)

The process is like the former, except isopropyl chloride is concentrated by distillation. Low grade, raw feedstock is fed into a distillation tower. Substantially all of the isopropyl chloride and most of the chloropropene content of the tower feed is recovered in the tower overhead fraction. Substantially all of the heavier compounds contained in the tower feed are recovered in the tower bottoms. There are two advantages to performing this step prior to the chlorination reaction. The first advantage is lower chlorine consumption—because some of the compounds that would compete with chloropropenes and consume chlorine are removed. The second advantage is smaller down stream equipment may be used, since it does not have to handle the heavy ends removed by the front-end distillation.

The preferred overhead operating pressure of the front-end distillation is 0 to 50 psig with 10-15 psig being the most preferred range. The bottom temperature should be kept below 160° C., preferably below 130° C. to prevent dehydrochlorination of the bottom material.

### EXAMPLE

The following composition is an example of a low-grade feedstock:

Propylene=1.5 wt %

Isopropyl chloride=38.6 wt %

Chloropropene isomers=6.9 wt %

1-chloropropane=2.0 wt %

Dichloropropane=49.9 wt %

Miscellaneous heavy ends=1.1 wt %

This composition is continuously fed to an agitated, jacketed, glass lined reactor. A chlorinating agent such as chlorine is continuously fed to the reactor. The chlorine feed rate is 105 mole percent of the olefin feed rate to the tower (5% excess chlorine). The olefins are propylene and chloropropene isomers shown in the composition example for low-grade feedstock. Reactor temperature is maintained at 38° C., by circulating tempered water through the reactor's jacket. Reactor residence time is 5.2 hours.

At these conditions, 99.1% of the chlorine fed to the reactor is consumed. Chloropropene isomer conversion is 99.9%. Isopropyl chloride losses, due to substitution reactions, are 9.9%. Reactor pressure, if no venting is allowed, is near 50 psig.

The reactor effluent is heated to 55° C. and passed through a plug flow reactor with a residence time of 5 min. Chlorine conversion is driven near toward completion. The effluent of the second reactor is fed into the first of two distillation towers.

The first distillation tower recovers substantially all of the hydrogen chloride in the overhead fraction. Substantially all of the organic compounds, including isopropyl chloride are recovered in the bottom fraction. Tower overhead pressure in this example is 50 psig. The tower bottom temperature is near 96° C.

The bottom fraction of this tower is fed to another distillation unit. This unit is operated at 5 psig overhead pressure and has a bottom temperature near 120° C. Isopropyl chloride is recovered in the tower overhead fraction. The purity of the isopropyl chloride recovered in this example is 99.5%.

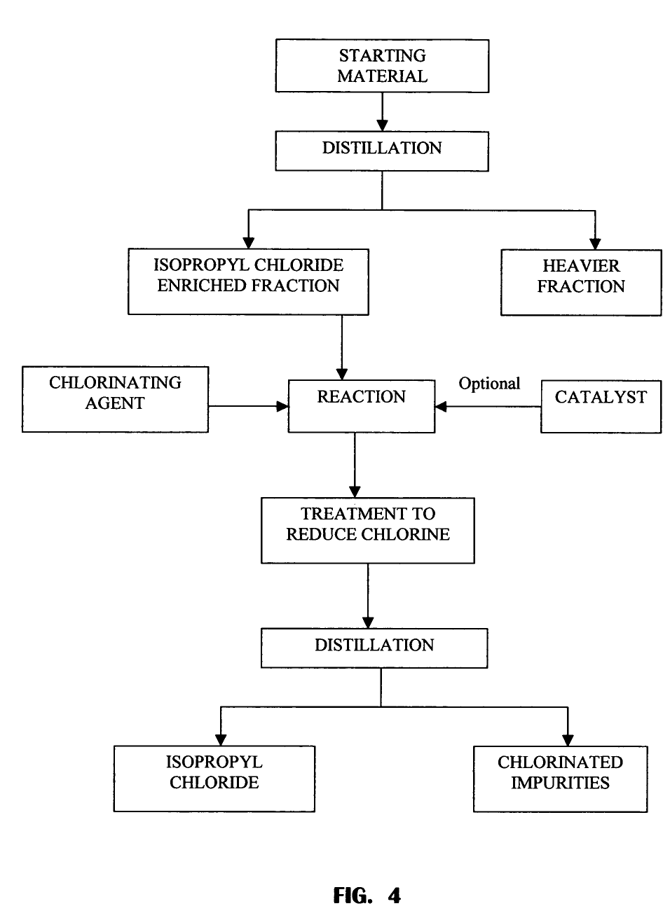

Description of a Process Using Treatment to Remove Excess Chlorine (FIG. 3)

This process is similar to the previously described process which has no front-end distillation except that after the reaction of the starting materials with the chlorinating agent, the reaction mixture is treated to remove or reduce the concentration of residual chlorine. This treatment acts to protect the downstream distillation equipment from excessive corrosion. To this end, any effective means may be employed. For example, the chlorinated mixture may be treated with stoichiometric qualities of a metal sulfite (i.e., sodium sulfite, potassium sulfite), sulfur dioxide, a “per” compound (that is a compound having an element in a high state of oxidation such as hydrogen peroxide, sodium percarbonate), an easily separated olefin such as propylene or olefin having a longer chain of carbon atoms. Similar methods would apply if the starting material contains impurities that react with chlorine relatively more easily than isopropyl chloride does. For example, alkenes of any kind, alkynes of any kind, etc. Following the treatment to remove excess chlorine, the treated reaction mixture is distilled as previously described with either the two tower or single vessel procedure.

The conditions of pressure, temperature and time are as described in the above procedures.

Description of a Process Using Front-End Distillation and Treatment to Remove Excess Chlorine (FIG. 4)

This procedure is similar to the above-described method using the treatment but has a further step of including an initial distillation to enrich the isopropyl fraction before reacting the enriched fraction with a chlorinating agent. After the reaction, which may also include the introduction of a catalyst as previously described, the procedure is to treat the reaction mixture with materials as described above. Distillation follows the treatment to obtain the highly purified isopropyl chloride. The distillation may include at least two towers or may be in a single vessel.

The conditions of temperature, pressure and time are as described in the above procedures.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A method to prepare purified isopropyl chloride from a starting material containing isopropyl chloride and impurities that are relatively easy to oxidize compared to isopropyl chloride, which comprises the steps of:
a) reaction of a chlorinating agent with the starting material to produce a mixture containing isopropyl chloride and chlorinated impurities,
b) distillation to separate isopropyl chloride from the chlorinated impurities.

2. The method of claim 1, wherein the impurities that are relatively easy to oxidize compared to isopropyl chloride are primarily olefins.

3. The method of claim 1, wherein a catalyst is employed to improve the chlorination kinetics.

4. The method of claim 3, wherein the catalyst is selected from the group consisting of light, ferric chloride and other transition metal chlorides, aluminum chlorides, antimony chlorides, stannic chloride, zinc chloride, lewis acids, bismuth chloride, gallium chloride, boron chloride, activated carbon, alumina and silica.

5. The method of claim 1, wherein the chlorination step is carried out in the liquid phase.

6. The method of claim 1, wherein the chlorination step is carried out in the vapor phase.

7. The method of claim 1, wherein excess chlorine in the range of 1%-35% is added to the starting material.

8. The method of claim 7, wherein the excess chlorine is added in a preferred range of 5%-15%.

9. The method of claim 1, wherein the reaction temperature is between 0° C. and 60° C.

10. The method of claim 9, wherein the preferred reaction temperature is between 10° C. and 30° C.

11. The method of claim 1, wherein the distillation is performed in two towers, in the first tower, hydrogen chloride is separated from isopropyl chloride, in the second tower, isopropyl chloride is separated from heavier chlorinated hydrocarbons.

12. The method of claim 1, wherein the chlorination and distillation are performed in a single vessel.

13. The method of claim 12, wherein the starting material and the chlorinating agent are added to the single vessel near the bottom of the single vessel.

14. The method of claim 12, wherein the distillation has an operating pressure of 0-50 psig.

15. The method of claim 12, wherein the distillation is conducted with the vessel having a bottom temperature below 160° C.

16. The method of claim 12, wherein the starting material and chlorinating agent are premixed before being introduced near the bottom of the single vessel.

17. Isopropyl chloride having a purity of approximately 99.5% prepared by the method of claim 1.

18. A method to prepare purified isopropyl chloride from a starting material containing isopropyl chloride and impurities that are relatively easy to oxidize compared to isopropyl chloride, which comprises the steps of:
a) distillation of the starting material to produce a fraction enriched in isopropyl chloride,
b) reaction of a chlorinating agent with the enriched fraction to produce a mixture containing isopropyl chloride and chlorinated impurities,
c) distillation of the mixture from step b) to separate purified isopropyl chloride from the chlorinated impurities.

19. The method of claim 18, wherein the impurities that are relatively easy to oxidize compared to isopropyl chloride are primarily olefins.

20. The method of claim 18, wherein a catalyst is employed to improve the chlorination kinetics.

21. The method of claim 20, wherein the catalyst is selected from the group consisting of light, ferric chloride and other transition metal chlorides, aluminum chlorides, antimony chlorides, stannic chloride, zinc chloride, lewis acids, bismuth chloride, gallium chloride, boron chloride, activated carbon, alumina and silica.

22. The method of claim 18, wherein the chlorination step is carried out in the liquid phase.

23. The method of claim 18, wherein the chlorination step is carried out in the vapor phase.

24. the method of claim 18, wherein the distillation of the starting material is conducted at an operating pressure of 0-50 psig.

25. The method of claim 24, wherein a preferred operating pressure is 10-15 psig.

26. The method of claim 18, wherein the distillation of the starting material has a bottom temperature less than 160° C.

27. The method of claim 18, wherein the reaction is maintained of approximately 38° C.

28. A method to prepare purified isopropyl chloride from a starting material containing isopropyl chloride and impurities that are relatively easy to oxidize compared to isopropyl chloride, which comprises the steps of:
a) reaction of a chlorinating agent with the starting material to produce a mixture containing isopropyl chloride and chlorinated impurities,
b) treatment of the product mixture from step a) to produce a treated mixture containing reduced chlorine concentration,
c) distillation of the treated product mixture from step b) to separate purified isopropyl chloride from the chlorinated impurities.

29. The method of claim 28, wherein the impurities that are relatively easy to oxidize compared to isopropyl chloride are primarily olefins.

30. The method of claim 28, wherein the product mixture is treated with a substance selected from the group consisting of a metal sulfite, sulfur dioxide, a “per” compound and an easily separated olefin.

31. The method of claim 26, wherein a catalyst is employed to improve the chlorination kinetics.

32. The method of claim 29, wherein the catalyst is selected from the group consisting of light, ferric chloride and other transition metal chlorides, aluminum chlorides, antimony chlorides, stannic chloride, zinc chloride, lewis acids, bismuth chloride, gallium chloride, boron chloride, activated carbon, alumina and silica.

33. The method of claim 28, wherein the chlorination step is carried out in the liquid phase.

34. The method of claim 28, wherein the chlorination step is carried out in the vapor phase.

35. A method to prepare purified isopropyl chloride from a starting material containing isopropyl chloride and impurities that are relatively easy to oxidize compared to isopropyl chloride, which comprises:
a) distillation of the starting material to produce a fraction enriched in isopropyl chloride,
b) reaction of a chlorinating agent with the enriched fraction to produce a mixture containing isopropyl chloride and chlorinated impurities,
c) treatment of the product mixture from step b) to produce a mixture containing reduced chlorine concentration,
d) distillation of the treated mixture from step c) to separate purified isopropyl chloride from the chlorinated impurities.

36. The method of claim 35, wherein the impurities that are relatively easy to oxidize compared to isopropyl chloride are primarily olefins.

37. The method of claim 35, wherein a catalyst is employed to improve the chlorination kinetics.

38. The method of claim 37, wherein the catalyst is selected from the group consisting of light, ferric chloride and other transition metal chlorides, aluminum chlorides, antimony chlorides, stannic chloride, zinc chloride, lewis acids, bismuth chloride, gallium chloride, boron chloride, activated carbon, alumina and silica.

39. The method of claim 35, wherein the chlorination step is carried out in the liquid phase.

40. the method of claim 35, wherein the chlorination step is carried out in the vapor phase.
